(19) United States
(12) Patent Application Publication
(10) Pub. No.: US 2009/0124636 A1
(43) Pub. Date: May 14, 2009

(54) CHEMICAL COMPOUNDS

(75) Inventors: Christopher Gordon Barber, Kent (GB); David Cameron Pryde, Kent (GB); David Roy Fenwick, Kent (GB); Peter Thomas Stephenson, Kent (GB)

Correspondence Address: PFIZER INC;Steve T. Zelson, 150 EAST 42ND STREET, 5TH FLOOR - STOP 49, NEW YORK, NY, 10017-5612, US

(73) Assignee: PFIZER INC.

(21) Appl. No.: 12/296,683

(22) PCT Filed: Mar. 30, 2007
(86) PCT No.: PCT/IB2007/000978

§ 371 Date: Nov. 11, 2008

Related U.S. Application Data
(60) Provisional application No. 60/791,857, filed on Apr. 12, 2006.

(51) Int. Cl. A61K 31/506; C07D 401/02; A61K 31/454; C07D 401/14; A61K 31/437; A61P 31/18; A61P 31/12; C07D 221/02; A61K 31/444; C07D 239/24

(52) U.S. Cl. 514/256; 546/208; 514/326; 546/194; 514/318; 544/335; 546/125; 514/304

(57) ABSTRACT

The present invention provides compounds of formula (I) wherein R¹ to R⁶ and m are as defined hereinabove. The compounds of the present invention are modulators, especially antagonists, of the activity of chemokine CCR5 receptors. Modulators of the CCR5 receptor may be useful in the treatment of various inflammatory diseases, autoimmune diseases, pain, and. in the treatment of infection by HIV and genetically related retroviruses.

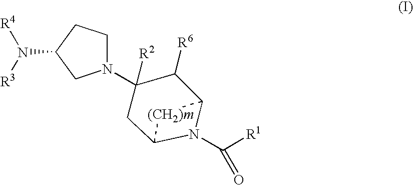

This invention relates to pyrrolidine piperidine derivatives, to processes and intermediates for their preparation, to compositions containing them and to their use.

More particularly, the present invention relates to the use of pyrrolidine piperidine derivatives in the treatment of a variety of disorders, including those in which the modulation of chemokine CCR5 receptors is implicated. Accordingly, the compounds of formula (I) are in particular useful in the treatment of HIV, such as HIV-1, and genetically related retroviral infections (and the resulting acquired immune deficiency syndrome, AIDS), inflammatory diseases, autoimmune diseases and pain.

The name “chemokine”, is a contraction of “chemotactic cytokines”. The chemokines comprise a large family of proteins which have in common important structural features and which have the ability to attract leukocytes. As leukocyte chemotactic factors, chemokines play an indispensable role in the attraction of leukocytes to various tissues of the body, a process which is essential for both inflammation and the body's response to infection. Because chemokines and their receptors are central to the pathophysiology of inflammatory and infectious diseases, agents which are active in modulating, preferably antagonizing, the activity of chemokines and their receptors, are useful in the therapeutic treatment of such inflammatory and infectious diseases.

The chemokine receptor CCR5 is of particular importance in the context of treating inflammatory and infectious diseases. CCR5 is a receptor for chemokines, especially for the macrophage inflammatory proteins (MIP) designated MIP-1α and MIP-1β, and for a protein which is regulated upon activation and is normal T-cell expressed and secreted (RANTES).

It is desirable to provide compounds for treatment of HIV and other indications which have one or more of the following properties: are selective, have a rapid onset of action, are potent, are stable, are resistant to metabolism, or have other desirable drug-like properties.

We have now found a group of compounds which are potent modulators, in particular antagonists, of the CCR5 receptor.

According to a first aspect of the invention there is provided a compound of formula (I)

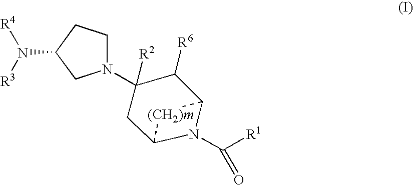

or a pharmaceutically acceptable salt, solvate of derivative thereof, wherein:

R¹ is aryl; or Het¹; and wherein the said aryl and Het¹ are substituted by 0 to 3 atoms or groups selected from C₁₋₆ alkyl, C₃₋₇ cycloalkyl, C₁₋₆ alkoxy, C₁₋₆ alkoxyC₁₋₆ alkyl, halogen, C₁₋₆ haloalkyl, OH, CN, phenyl or imidazolyl;

R² is H or C₁₋₃ alkyl

R³ is C₁₋₆alkyl, C₃₋₇cycloalkyl, aryl, arylC₁₋₃alkyl, Het²C₁₋₃alkyl wherein the said aryl and Het² are substituted by 0 to 3 atoms or groups selected from C₁₋₆ alkyl, C₃₋₇ cycloalkyl, C₁₋₆ alkoxy, C₁₋₆ alkoxyC₁₋₆ alkyl, halogen, C₁₋₆ haloalkyl, OH or CN;

R⁴ is COR⁵ or SO₂R⁵;

R⁵ is H, aryl, arylC₁₋₃alkyl, C₁₋₆ alkyl, C₃₋₇ cycloalkyl, C₃₋₇cycloalkylC₁₋₃alkyl, C₁₋₆ alkoxy, C₁₋₆ alkoxyC₁₋₆ alkyl, C₀₋₆alkylaminoC₀₋₆alkyl, or a 5 to 6 membered saturated heterocycle containing one to three heteroatoms selected from N, O and S (such as tetrahydrofuran or tetrahydropyran); wherein the said C₁₋₆ alkyl, C₃₋₇ cycloalkyl, C₃₋₇cycloalkylC₁₋₃alkyl, C₁₋₆ alkoxy, C₁₋₆ alkoxyC₁₋₆ alkyl and C₀₋₆alkylaminoC₀₋₆alkyl are substituted by 0 to 3 atoms or groups selected from halogen, C₁₋₆ alkoxy or OH;

R⁶ is H or CH₃;

m is 0, 1, 2 or 3;

“-----” represents an optional C—C bond forming an alkylene bridge;

Het¹ is a 5 to 10-membered aromatic heterocycle containing one to three heteroatoms selected from N, O and S, and wherein when Het¹ is a N-containing heterocycle, N-oxides thereof;

Het² is a 5 or 6 membered aromatic heterocycle containing one to three heteroatoms selected from N, O and S, and wherein when Het¹ is a N-containing heterocycle, N-oxides thereof.

In the above definitions, aryl means phenyl or napthyl. Halogen means fluorine chlorine, bromine or iodine. Alkyl moieties containing the requisite number of carbon atoms can be straight chain or branched. Examples of alkyl include methyl, ethyl, n-propyl, i-propyl, n-butyl, sec-butyl and t-butyl. Examples of alkoxy include methoxy, ethoxy, n-propoxy, i-propoxy, n-butoxy, sec-butoxy and t-butoxy. Examples of cycloalkyl include cyclopropyl, cyclobutyl, cyclopentyl, cyclohexyl and cycloheptyl.

In one embodiment R¹ is phenyl or Het¹, wherein Het¹ is a 5 to 6 membered aromatic heterocycle containing 1 to 3 heteroatoms selected from N, O and S, and wherein when Het¹ is a N-containing heterocycle, N-oxides thereof; wherein said phenyl and Het¹ are substituted by 0 to 3 atoms or groups selected from C₁₋₆ alkyl, C₃₋₇ cycloalkyl, C₁₋₆ alkoxy, C₁₋₆ alkoxyC₁₋₆ alkyl, halogen, C₁₋₆ haloalkyl, OH, CN, phenyl or imidazolyl;

In yet a further embodiment R¹ is phenyl, pyridyl, pyrimidyl, pyridyl N-oxide or pyrimidyl N-oxide, pyrazolyl, oxazolyl or isoxazolyl substituted by 0 to 3 atoms or groups selected from C₁₋₆ alkyl, C₃₋₇ cycloalkyl, C₁₋₆ alkoxy, C₁₋₆ alkoxyC₁₋₆ alkyl, halogen, C₁₋₆ haloalkyl, OH, CN, phenyl or imidazolyl.

In yet a further embodiment R¹ is phenyl, pyridyl, pyrimidyl, pyridyl N-oxide or pyrimidyl N-oxide, substituted with 0 to 2 atoms or groups selected from C₁₋₃alkyl, C₁₋₆ alkoxy or halogen.

In yet a further embodiment R¹ is phenyl, pyridyl, pyrimidyl, pyridyl N-oxide or pyrimidyl N-oxide mono or disubstituted at the ortho position relative to the carbon attached to the adjacent carbonyl of formula (I) wherein the substituents are selected from C₁₋₃alkyl or halogen. Thus dimethyl substitution on phenyl would give 2,6 dimethyl substitution, as shown in the examples.

In yet a further embodiment R¹ is phenyl substituted as in any of the embodiments above.

In yet a further embodiment R¹ is 2,6-dimethylphenyl, 2,4-dimethylpyridin-3-yl or 4,6-dimethylpyrimidin-5-yl.

In yet a further embodiment R² is H.

In yet a further embodiment R³ is benzyl, pyridylmethyl or pyrimidylmethyl substituted by 0 to 3 atoms or groups selected from C₁₋₆ alkyl, C₃₋₇ cycloalkyl, C₁₋₆ alkoxy, C₁₋₆ alkoxyC₁₋₆ alkyl, halogen, C₁₋₆ haloalkyl, OH or CN.

In yet a further embodiment R³ is benzyl substituted by 0 to 3 atoms or groups selected from C₁₋₆ alkyl, C₃₋₇ cycloalkyl, C₁₋₆ alkoxy, C₁₋₆ alkoxyC₁₋₆ alkyl, halogen, C₁₋₆ haloalkyl, OH or CN.

In yet a further embodiment R³ is benzyl substituted by 0 to 2 atoms or groups selected from C₁₋₃alkyl, halogen, C₁₋₃alkoxy, or C₁₋₃haloalkyl.

In yet a further embodiment R³ is benzyl substituted by 0 to 2 atoms selected from fluorine or chlorine.

In yet a further embodiment R⁴ is COR⁵ or SO₂R⁵ and R⁵ is H, phenyl, C₁₋₆ alkyl, C₃₋₇cycloalkyl, C₃₋₇cycloalkylmethyl, C₁₋₃ alkoxy, C₁₋₆ alkoxyC₁₋₃ alkyl or C₁₋₆alkylamino wherein the said C₁₋₆ alkyl, C₃₋₇ cycloalkyl, C₃₋₇cycloalkylmethyl, C₁₋₃ alkoxy, C₁₋₃alkoxyC₁₋₆ alkyl, and C₁₋₆alkylamino are substituted by 0 to 3 atoms or groups selected from halogen, C₁₋₆ alkoxy or OH.

In yet a further embodiment R⁴ is COR⁵ or SO₂R⁵ and R⁵ is H, phenyl, C₁₋₆ alkyl, C₃₋₇cycloalkyl, C₃₋₇cycloalkylmethyl, C₁₋₃ alkoxy, C₁₋₆ alkoxyC₁₋₃ alkyl or C₁₋₆alkylamino wherein the said C₁₋₆ alkyl, C₃₋₇ cycloalkyl, C₃₋₇cycloalkylmethyl, C₁₋₃ alkoxy, C₁₋₃alkoxyC₁₋₆ alkyl, and C₁₋₆alkylamino substituted by 0 to 3 halogen atoms.

In yet a further embodiment R⁴ is COR⁵ or SO₂R⁵ and R⁵ is C₃₋₇ cycloalkyl, C₁₋₃ alkoxyC₁₋₃ alkyl or C₁₋₄ alkylamino wherein the cycloalkyl is substituted with 0 to 2 fluorine atoms.

In yet a further embodiment R⁴ is COR⁵ or SO₂R⁵ and R⁵ is C₃₋₇ cycloalkyl which is difluoro substituted on the same ring carbon (such as 3,3-difluorocyclobutyl).

In yet a further embodiment R⁴ is COR⁵, wherein R⁵ is as defined and optionally substituted as in any preceding embodiment.

In yet a further embodiment R⁶ is H.

In yet a further embodiment m is 0 or 2.

In yet a further embodiment m is 2 and forms an alkylene bridge.

In yet a further embodiment m is 0.

It is to be understood that the invention covers all combinations of particular embodiments of the invention as described hereinabove, consistent with the definition of the compounds of formula (I).

The invention includes the compounds of formula (I) and pharmaceutically acceptable salts, solvates or derivatives thereof (wherein derivatives include complexes, prodrugs, polymorphs and crystal habits thereof, and isotopes, as well as salts and solvates thereof) and reference to compounds of formula (I) should be construed accordingly.

Pharmaceutically acceptable salts of the compounds of formula (I) include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002), incorporated herein by reference.

The compounds of formula (I) may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of formula (I) may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of formula (I) may also exist in multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004), incorporated herein by reference. For a general review of multi-component complexes, see J Pharm Sci, 64 (8), 1269-1288, by Haleblian (August 1975), incorporated herein by reference.

The compounds of formula (I) may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970), incorporated herein by reference.

As indicated, so-called ‘prodrugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association), both incorporated herein by reference.

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985), incorporated herein by reference.

Moreover, certain compounds of formula (I) may themselves act as prodrugs of other compounds of formula (I).

Also included within the scope of the invention are metabolites of compounds of formula (I), that is, compounds formed in vivo upon administration of the drug. Some examples of metabolites in accordance with the invention include:

(i) where the compound of formula (I) contains a methyl group, an hydroxymethyl derivative thereof (—CH₃->—CH₂OH);
(ii) where the compound of formula (I) contains an alkoxy group, an hydroxy derivative thereof (—OR->—OH);
(iii) where the compound of formula (I) contains a tertiary amino group, a secondary amino derivative thereof (—NR¹R²->—NHR¹ or —NHR²);
(iv) where the compound of formula (I) contains a secondary amino group, a primary derivative thereof (—NHR¹->—NH₂);
(v) where the compound of formula (I) contains a phenyl moiety, a phenol derivative thereof (-Ph->-PhOH); and
(vi) where the compound of formula (I) contains an amide group, a carboxylic acid derivative thereof (—CONH₂->COOH).

Compounds of formula (I) contain one or more asymmetric carbon atoms and therefore exist as two or more stereoisomers. The asymmetric carbon on the pyrrolidine ring, as shown in formula (I), shows the R-configuration. When R⁶ is C₁₋₄alkyl, a further asymmetric carbon exists, as illustrated in example 60, at the carbon connected to R⁶. Compounds of formula (I) wherein m≠0, i.e., which contain a bridged piperidine ring, can be in either endo- or exo-configuration, and therefore geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, a keto, or oxime group, or so-called valence tautomerism in compounds which contain an aromatic moiety.

Compounds of formula (I) may exhibit atropisomerism, or axial chirality, which occurs when molecules are chiral by virtue of their overall shape rather than having chiral centres. The 3D shape which renders these molecules chiral is maintained as a result of hindered rotation around a bond or bonds. Free rotation about a single covalent bond is impeded sufficiently that interconversion of the stereoisomeric conformations (atropisomers) is slow enough to allow separation and isolation under predetermined conditions. The energy barrier to thermal racemization may be determined by the steric hindrance to free rotation of one or more bonds forming a chiral axis. It follows that a single compound may exhibit more than one type of isomerism.

Included within the scope of the present invention are all stereoisomers of the compounds of formula (I), including all optical isomers, geometric isomers, atropisomers and tautomeric forms as well as compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Endo/exo and cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

The present invention also includes all pharmaceutically acceptable isotopically-labelled compounds of formula (I) wherein one or more atoms are replaced by atoms having the same atomic number, but an atomic mass or mass number different from the atomic mass or mass number which predominates in nature.

Preferred compounds of formula (I) include the examples, particularly examples 1 to 55 and 58 to 72, and pharmaceutically acceptable salts, solvates and derivatives thereof.

In the general processes, and schemes, that follow: R¹, R², R³, R⁴, R⁵ and R⁶ are as previously defined unless otherwise stated; X is halo; Z is OH, or a carboxylic acid activating group such as halo, (suitably chloro) or 1H-imidazol-1-yl; Pg is an amino protecting group; BOC is tert-butoxycarbonyl; CBz is benzyloxycarbonyl; Bn is benzyl, Fmoc is 9-fluorenylmethoxycarbonyl; MeOH is methanol; EtOH is ethanol; EtOAc is ethyl acetate; Et₂O is diethyl ether; THF is tetrahydrofuran; DMSO is dimethyl sulfoxide; DCM is dichloromethane; AcOH is acetic acid; TFA is trifluoroacetic acid; STAB is sodium triacetoxyborohydride; DMA is N,N-dimethylacetamide; DMSO is dimethylsulphoxide; NMM is N-methylmorpholine; WSCDI is 1-(3-dimethylaminopropyl)-3-ethylcarbodiimide hydrochloride; DCC is N,N′-dicyclohexylcarbodiimide; HOBT is 1-hydroxybenzotriazole hydrate; PyBOP® is Benzotriazol-1-yloxytris(pyrrolidino)phosphonium hexafluorophosphate; PyBrOP® is bromo-tris-pyrrolidino-phosphonium; Hünig's base is N-ethyldiisopropylamine; Et₃N is triethylamine; HBTU is O-Benzotriazol-1-yl-N,N,N′,N′-tetramethyluronium hexafluorophosphate; Ti(ⁱOPR)₄ is titanium tetraisopropoxide.

The compound of formula (I) can be prepared by routes such as by the procedures described in the general process and Examples set out hereinafter. It will be appreciated that the present invention also encompasses any one of these processes for preparing compounds of formula (I) as well as any novel intermediates used in the processes.

Compounds of formula (I) wherein R² is H and m is 0 are prepared by reacting a compound of a compound of formula (III)

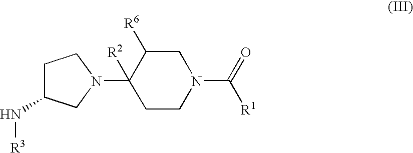

(a) with a compound of formula (II)

R⁵COZ (II)

wherein Z is OH or a carboxylic acid activating group or 1H-imidazol-1-yl; or
(b) with a compound of formula (XII)

R⁵S0₂X (XII)

wherein X is halogen; or
(c) with a compound of formula (XIII)

C₀₋₆alkyl NCO (XIII).

In step (c) the C₀₋₆alkyl N moiety is substituted as R⁵ onto the amine (NHR³) of formula (III).

This aspect of the invention is further illustrated in schemes 1 to 3.

Compounds of formula (I) wherein R² is C₁₋₃ alkyl and m is 0, can be prepared by reacting a compound of formula (XIV)

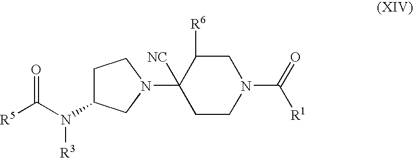

with a compound of formula (XV)

R²MgX (XV)

wherein X is a halogen.

This process is further illustrated in scheme 4, step (j).

Bridged compounds of formula (I) can be formed in accordance with scheme 5.

The schemes (1 to 5) which further illustrate the general methods for the preparation of the compounds of formula (I) and intermediates thereto, follow below.

It will be appreciated by those skilled in the art that, as illustrated in the schemes that follow, it may be necessary or desirable at any stage in the synthesis of compounds of formula (I) to protect one or more sensitive groups in the molecule so as to prevent undesirable side reactions. In particular, it may be necessary or desirable to protect amino groups. The protecting groups used in the preparation of compounds of formula (I) may be used in conventional manner. See, for example, those described in ‘Protective Groups in Organic Synthesis’ by Theodora W Green and Peter G M Wuts, third edition, (John Wiley and Sons, 1999), in particular chapter 7, pages 494-653 (“Protection for the Amino Group”), incorporated herein by reference, which also describes methods for the removal of such groups.

The amino protecting groups t-butoxycarbonyl (Boc), 9-fluorenylmethoxycarbonyl (Fmoc), benzyloxycarbonyl (Cbz), methylformate, benzyl and acetyl are of particular use in the preparation of compounds of formula (I) and intermediates thereto.

It will also be appreciated by those skilled in the art that certain of the procedures described in the schemes for the preparation of compounds of formula (I) or intermediates thereto may not be applicable to some of the possible substituents.

It will be further appreciated by those skilled in the art that it may be necessary or desirable to carry out the transformations described in the schemes in a different order from that described, or to modify one or more of the transformations, to provide the desired compound of formula (I).

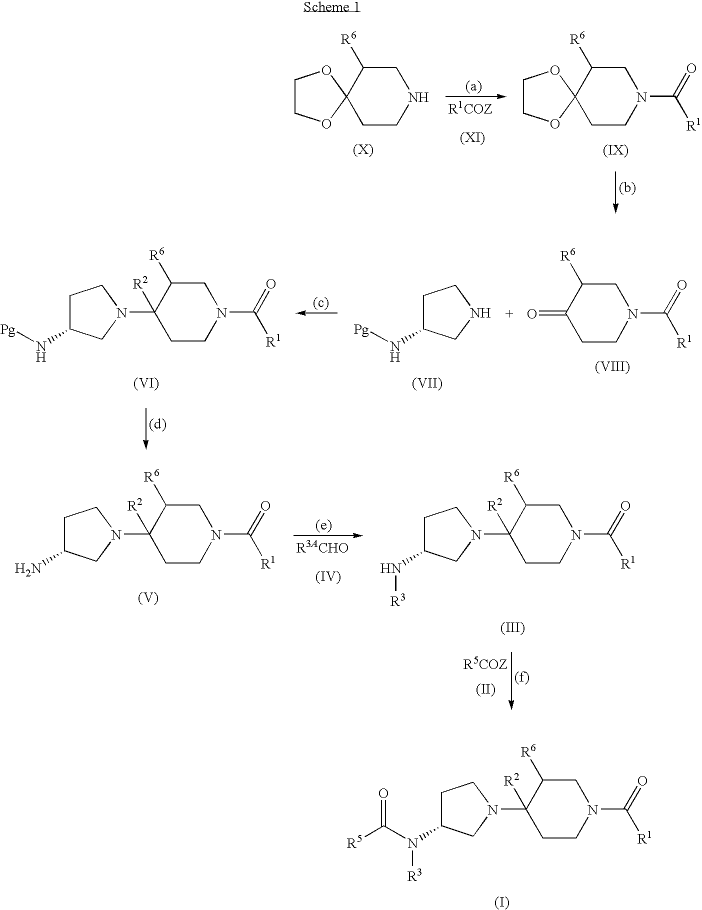

Scheme 1 illustrates the preparation of compounds of formula (I) when R² is H.

With specific reference to scheme 1 the transformations depicted therein may be effected as follows:

Step (a) Compounds of formula (IX) may be prepared by reacting compounds of formula (X) with compounds of formula (XI) under conventional acid amine coupling conditions. The acid amine coupling is conveniently effected using an amine of formula (X) and R⁵COZ of formula (XI), where Z is OH or a carboxylic, acid activating group such as halogen (suitably chorine) or 1H-imadidazol-1-yl; an excess of an acid acceptor, such as triethylamine or Hünig's base or an inorganic base such as potassium carbonate; in a solvent, such as a haloalkane (e.g. DCM).

Alternatively, the acid/amine coupling is effected using an acid of formula (XI) activated by activating reagents such as WSCDI or DCC and HOBt or HOAt; an excess of an acid acceptor such as triethylamine or N-ethyl-N,N-diisopropylamine; in a solvent such as NMM or DCM. Alternatively, PYBOP®/PyBrOP® or Mukaiyama's reagent may be used under standard conditions.

Step (b) Compounds of formula (VII) may be prepared from compounds of formula (IX) under conventional conditions of acidic hydrolysis.

Step (c) Compounds of formula (VI) may be prepared by reacting compounds of formula (VII) with compounds of formula (VII) under conventional reductive amination conditions. Conveniently, reductive amination may be effected by reacting compounds of formula (VII) with amines of formula (VII) in the presence of a reducing agent such as NaBH₄, Na(OAc)₃BH, NaCNBH₃; optionally in the presence of NaOAc or AcOH; optionally in the presence of an additive such as titanium tetraisopropoxide; further optionally in the presence of a drying agent such as MgSO₄ or molecular sieves; in a solvent such as DCM, methanol or DCE.

Step (d) Deprotection of compounds of formula (VI) may be undertaken using standard methodology. Preferred protecting groups include BOC whereupon deprotection may be effected using TFA or HCl in a solvent such as an ether (e.g. diethyl ether), a haloalkane (e.g. DCM) or ethyl acetate). Conveniently the reaction is performed at a temperature between 0° C. to RT. Alternative preferred protecting groups include Bn, CBz and Fmoc which may be deprotected by methods known to those skilled in the art.

Step (e) Compounds of formula (III) may be prepared by reacting compounds of formula (V) with compounds of formula (IV), wherein moiety R³AC of formula (IV) is incorporated as R³ into formula (III). This reaction may be effected according to the reductive amination conditions described above in step (c).

Step (f) Compounds of formula (I) may be prepared by reacting compounds of formula (III) with compounds of formula (II), wherein Z is as defined in step (a). This acid amine coupling may be effected according to the conditions described above in step (a).

Although Step (e) and step (f) are shown as two separate steps, they may conveniently be performed in a one-pot procedure.

Compounds of formula (I) wherein R⁴ is SO₂R⁵ may be prepared by methods which are directly analogous to preparation of compounds of formula (I) wherein R⁴ is COR⁵. In particular, compounds of formula (I) wherein R⁴ is SO₂R⁵ may be prepared according to Scheme I when the acid amine coupling step (f) is replaced by standard sulfonylation conditions known to those skilled in the art. Sulfonation may conveniently be effected according to Scheme 2.

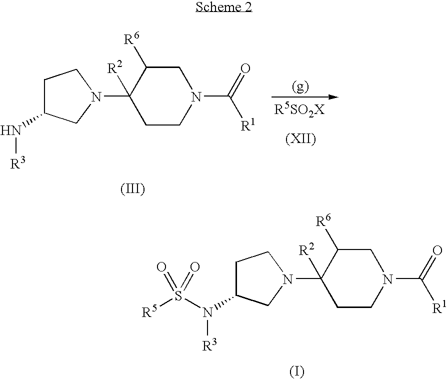

Step (g) Compounds of formula (I) wherein R⁴ is SO₂R⁵ may be prepared by reacting compounds of formula (III) with a sulfonylating agent such as a compound of formula (XII), R⁵SO₂X, wherein X is a halogen conveniently chlorine or fluorine.

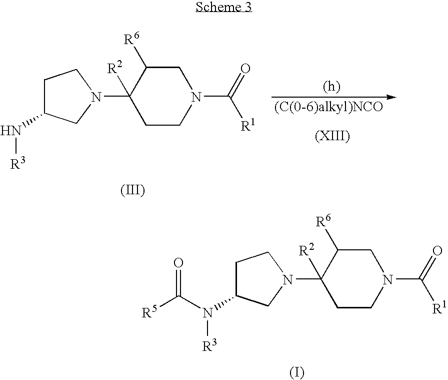

Step (h) Compounds of formula (I) wherein R⁴ is CONH C₀₋₆alkyl may be prepared by reacting compounds of formula (III) with an isocyanate such as a compound of formula (XIII), C₀₋₆alkylNCO.

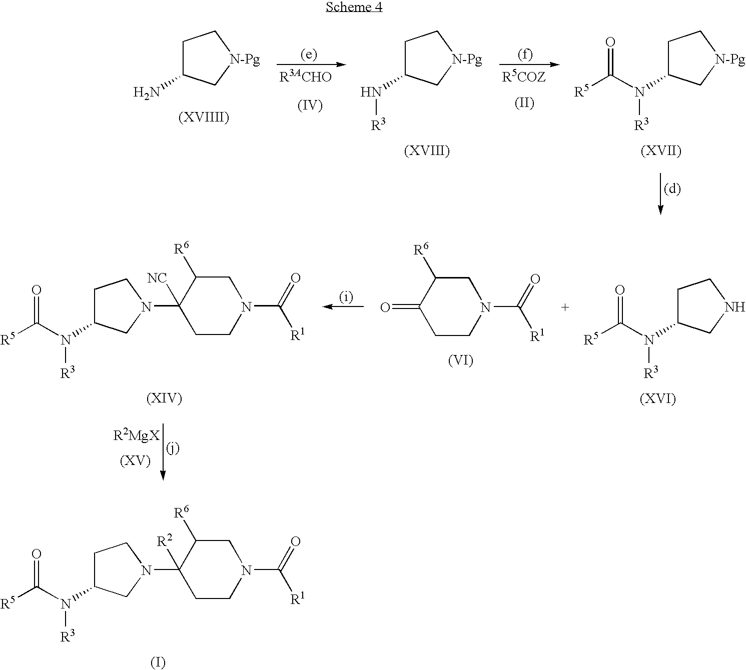

Scheme 4 illustrates the preparation of compounds of formula (I) when R² is C₁₋₃alkyl. Amines of formula (XVIII) can be prepared from compounds of formula (XVIIII) under conventional reductive amination conditions as set out in scheme 1 step (e). Thus moiety R^{3A}C of formula (IV) is incorporated as R³ into formula (XVIII).

Amides of formula (XVII) can be prepared by coupling an amine of formula (XVIII) with acid R⁵COZ under conventional coupling conditions as set out in scheme 1, step (f).

Compounds of formula (XVI) can be prepared by deprotection of compounds of formula (XVII) using standard methodology as set out in scheme 1, step (d).

Step (i): Compounds of formula (XIV) may be prepared by reacting compounds of formula (VI), with a compound of formula (XVI) in the presence of a suitable cyanating agent (e.g. Et₂AlCN (J. Am. Chem. Soc. 94 (13), 4635, 1972), acetone cyanohydrin, or an acid such as acetic acid, sulphuric acid, NaHSO₄, KHSO₃ or Na₂S₂O₅ and a cyanide source such as NaCN, KCN, trimethylsilylcyanide, glycolonitrile or dimethylaminoacetonitrile); optionally in the presence of Ti(ⁱOPr)₄; in a solvent such as a haloalkane (e.g. DCM or dichloroethane) or THF; at a temperature between 0° C. and 100° C. (e.g between 0° C. and 50° C., conveniently at ambient temperature)

Alternatively compounds of formula (XIV) may be generated by the action of HCN on the corresponding imine which may be either preformed or formed in situ from the reaction of a compound of formula (VI) and a compound of formula (XVI) in the presence of a solvent.

Step (j): Compounds of formula (XIV) may be converted to compounds of formula (I) via a Bruylants Reaction (e.g. C. Agami, F. Couty, G. Evano Organic Letters 2000, 14(2), 2085-2088). A compound of formula (I) may be prepared by reacting a compound of formula (XIV) with an organometallic agent such as a Grignard Reagent of formula (XV), R²MgBr, or an organolithium reagent of formula R²Li; optionally in the presence of trimethylaluminum; in a solvent such as THF or Et₂O; at a temperature between 0° C. and ambient. Conveniently an excess of Grignard Reagent may be used.

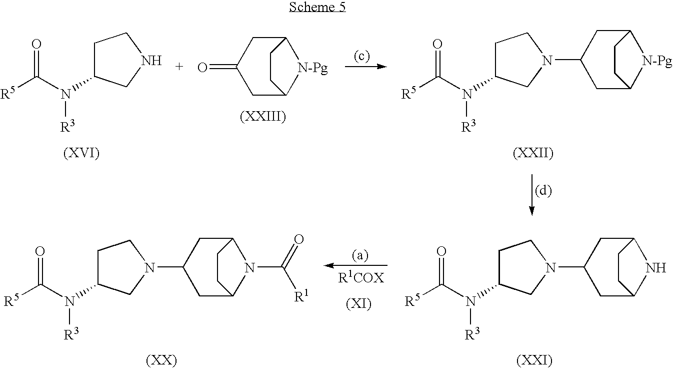

Compounds of formula (XXII) can be prepared from compounds of formula (XVI) and (XXIII) under conventional reduction amination conditions as set out in step (c) of scheme 1.

Compounds of formula (XXI) may be prepared by deprotecting compounds of formula (XXII) using standard methodology, as set out in step (d) scheme 1.

Compounds of formulae (VI), (X), (XVII) and (XXIII) are either commercially available or may be prepared by conventional chemistry.

The compounds of formula (I) and their pharmaceutically acceptable salts, solvates and derivatives are useful because they have pharmacological activity in animals, including humans. More particularly, they are useful in the treatment of a disorder in which the modulation, in particular antagonism of CCR5 receptors is implicated. Disease states of particular interest include HIV, retroviral infections genetically related to HIV and AIDS.

Other disease states of interest include inflammatory diseases, autoimmune diseases and pain.

The compounds of this invention may be used for treatment of respiratory disorders, including adult respiratory distress syndrome (ARDS), bronchitis, chronic bronchitis, chronic obstructive pulmonary disease, cystic fibrosis, asthma, emphysema, rhinitis, chronic sinusitis, sarcoidosis, farmer's lung, nasal polyposis, fibroid lung or idiopathic interstitial pneumonia.

Other conditions that may be treated are those triggered, affected or are in any other way correlated with T-cell trafficking in different organs. It is expected that the compounds of this invention may be useful for the treatment of such conditions and in particular, but not limited to, conditions for which a correlation with CCR5 or CCR5 chemokines has been established, and more particularly, but not limited to, the following: multiple sclerosis; Behcet's disease, Sjogren's syndrome or systemic sclerosis; arthritis, such as rheumatoid arthritis, spondyloarthropathies, gouty arthritis, osteoarthritis, systemic lupus erythematosus, and juvenile arthritis; and graft rejection, in particular, but not limited to, solid organ transplants, such as heart, lung, liver, kidney and pancreas transplants (e.g. kidney and lung allografts), and graft versus host rejection; inflammatory bowel disease, including Crohn's disease and ulcerative colitis; inflammatory lung conditions; endometriosis; renal diseases, such as glomerular disease (e.g. glomerulonephritis); fibrosis, such as liver, pulmonary and renal fibrosis; encephalitis, such as HIV encephalitis; chronic heart failure; myocardial infarction; hypertension; stroke; ischaemic heart disease; atherosclerotic plaque; restenosis; obesity; psoriasis; atopic dermatitis; CNS diseases, such as AIDS related dementias and Alzheimer's disease; anaemia; chronic pancreatitis; Hashimoto's thyroiditis; type I diabetes; cancer, such as non-Hodgkin's lymphoma, Kaposi's sarcoma, melanoma, multiple myeloma and breast cancer; pain, such as nociceptive pain and neuropathic pain (e.g. peripheral neuropathic pain); and stress response resulting from surgery, infection, injury or other traumatic insult.

Infectious diseases where modulation of the CCR5 receptor is implicated include acute and chronic hepatitis B Virus (HBV) and hepatitis C Virus (HCV) infection; bubonic, septicemic, and pneumonic plague; pox virus infection, such as smallpox; toxoplasmosis infection; mycobacterium infection; trypanosomal infection such as Chagas' Disease; pneumonia; and cytosporidiosis.

For a recent review of possible applications of chemokines and chemokine receptor blockers see Robeiro and Horuk, “The Clinical Potential of Chemokine Receptor Antagonists”, Pharmacology and Therapeutics 107 (2005) p 44-58.

Accordingly, in another aspect the invention provides a compound of formula (I) or a pharmaceutically acceptable salt, solvate or derivative thereof for use as a medicament.

In another aspect the invention provides a compound of formula (I) or a pharmaceutically acceptable salt, solvate or derivative thereof, for the treatment of a disorder in which the modulation of CCR5 receptors is implicated.

In another aspect the invention provides the use of a compound of formula (I) or of a pharmaceutically acceptable salt, solvate or derivative thereof, in the manufacture of a medicament for the treatment of a disorder in which the modulation of CCR5 receptors is implicated.

In another aspect the invention provides a method of treatment of a disorder in which the modulation of CCR5 receptors is implicated which comprises administering to a patient in need thereof (e.g a human patient or an animal patient) a therapeutically effective amount of a compound of formula (I) or a pharmaceutically acceptable salt, solvate or derivative thereof.

The compounds of formula (I) are useful in the treatment of the diseases, disorders or conditions mentioned above. Diseases of particular interest include HIV, retroviral infections genetically related to HIV and AIDS.

Further diseases of interest are inflammatory diseases, autoimmune diseases and pain.

Further diseases of interest are rheumatoid arthritis, graft rejection, fibrosis and pain.

For the avoidance of doubt, references herein to “treatment” include references to curative, palliative and prophylactic treatment.

Compounds of formula (I) intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of formula (I) or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form. Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995), incorporated herein by reference.

Suitable modes of administration include oral, parenteral, topical, inhaled/intranasal, rectal/intravaginal, and ocular/aural administration.

The compounds of formula (I) may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of formula (I) may also be used in fast-dissolving, fast-disintegrating dosage forms such as those described in Expert Opinion in Therapeutic Patents, 11 (6), 981-986, by Liang and Chen (2001), incorporated herein by reference.

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980), incorporated herein by reference.

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864, incorporated herein by reference. Details of other suitable release technologies such as high energy dispersions and osmotic and coated particles are to be found in Pharmaceutical Technology On-line, 25(2), 1-14, by Verma et al (2001), incorporated herein by reference. The use of chewing gum to achieve controlled release is described in WO 00/35298, incorporated herein by reference.

The compounds of formula (I) may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of formula (I) may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic)acid (PGLA) microspheres.

The compounds of formula (I) may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999), incorporated herein by reference.

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of formula (I) can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 g to 10 mg of the compound of the invention. The overall daily dose will typically be in the range 1 μg to 200 mg which may be administered in a single dose or, more usually, as divided doses throughout the day.

The compounds of formula (I) may be administered rectally or vaginally, for example, in the form of a suppository, pessary, vaginal ring or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of formula (I) may also be administered directly to the eye or ear, typically in the form of drops of a micronised suspension or solution in isotonic, pH-adjusted, sterile saline. Other formulations suitable for ocular and aural administration include ointments, gels, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of formula (I) may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148, incorporated herein by reference.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, having a weight of about 65 to 70 kg, the total daily dose of a compound of the invention is typically in the range 1 to 10,000 mg, such as 10 to 1,000 mg, for example 25 to 500 mg, depending, of course, on the mode of administration, the age, condition and weight of the patient, and will in any case be at the ultimate discretion of the physician. The total daily dose may be administered in single or divided doses.

Accordingly in another aspect the invention provides a pharmaceutical composition comprising a compound of formula (I) or a pharmaceutically acceptable salt, solvate or derivative thereof together with one or more pharmaceutically acceptable excipients, diluents or carriers.

The compounds of formula (I) and their pharmaceutically acceptable salts, solvates and derivatives may be administered alone or as part of a combination therapy. Thus included within the scope of the present invention are embodiments comprising co-administration of, and compositions which contain, in addition to a compound of the invention, one or more additional therapeutic agents.

Such multiple drug regimens, often referred to as combination therapy, may be used in the treatment and prevention of any of the diseases or conditions mediated by or associated with CCR5 chemokine receptor modulation, particularly infection by human immunodeficiency virus, HIV. The use of such combination therapy is especially pertinent with respect to the treatment and prevention of infection and multiplication of the human immunodeficiency virus, HIV, and related pathogenic retroviruses within a patient in need of treatment or one at risk of becoming such a patient. The ability of such retroviral pathogens to evolve within a relatively short period of time into strains resistant to any monotherapy which has been administered to said patient is well known in the literature. A recommended treatment for HIV is a combination drug treatment called Highly Active Anti-Retroviral Therapy, or HAART. HAART combines three or more HIV drugs. Thus, the methods of treatment and pharmaceutical compositions of the present invention may employ a compound of the invention in the form of monotherapy, but said methods and compositions may also be used in the form of combination therapy in which one or more compounds of formula (I) are co-administered in combination with one or more additional therapeutic agents such as those described in detail further herein.

The therapeutic agents that may be used in combination with the compounds of the present invention include, but are not limited to, those useful as HIV protease inhibitors (PIs), non-nucleoside reverse transcriptase inhibitors (NNRTIs), nucleoside/nucleotide reverse transcriptase inhibitors (NRTIs), CCR5 antagonists, agents which inhibit the interaction of gp120 with CD4, other agents which inhibit the entry of HIV into a target cell, inhibitors of HIV integrase, RNaseH inhibitors, prenylation inhibitors, maturation inhibitors which act by interfering with production of the HIV capsid protein, compounds useful as anti-infectives, and others as described below.

It will be appreciated by a person skilled in the art, that a combination drug treatment, as described herein above, may comprise two or more compounds having the same, or different, mechanism of action. Thus, by way of illustration only, a combination may comprise a compound of the invention and: one or more NRTIs; one or more NRTIs and a PI; one or more NRTIs and another CCR5 antagonist; a PI; a PI and an NNRTI; an NNRTI; and so on.

Examples of P is include, but are not limited to, amprenavir (141W94), CGP-73547, CGP-61755, DMP-450 (mozenavir), nelfinavir, ritonavir, saquinavir (invirase), lopinavir, TMC-126, atazanavir, palinavir, GS-3333, KN I-413, KNI-272, LG-71350, CGP-61755, PD 173606, PD 177298, PD 178390, PD 178392, U-140690, ABT-378, DMP-450, AG-1776, MK-944, VX-478, indinavir, tipranavir, TMC-114, DPC-681, DPC-684, fosamprenavir calcium (Lexiva), benzenesulfonamide derivatives disclosed in WO 03/053435, R-944, Ro-03-34649, VX-385, GS-224338, OPT-TL3, PL-100, PPL-100, SM-309515, AG-148, DG-35-VIII, DMP-850, GW-5950X, KNI-1039, L-756423, LB-71262, LP-130, RS-344, SE-063, UIC-94-003, Vb-19038, A-77003, BMS-182193, BMS-186318, SM-309515, JE-2147, GS-9005.

Examples of NRTIs include, but are not limited to, abacavir, GS-840, lamivudine, adefovir dipivoxil, beta-fluoro-ddA, zalcitabine, didanosine, stavudine, zidovudine, tenofovir disoproxil fumarate, amdoxovir (DAPD), SPD-754, SPD-756, racivir, reverset (DPC-817), MIV-210 (FLG), beta-L-Fd4C (ACH-126443), MIV-310 (alovudine, FLT), dOTC, DAPD, entecavir, GS-7340, emtricitabine (FTC).

Examples of NNRTIs include, but are not limited to, efavirenz, HBY-097, nevirapine, TMC-120 (dapivirine), TMC-125, etravirine, delavirdine, DPC-083, DPC-961, capravirine, rilpivirine, 5-{[3,5-Diethyl-1-(2-hydroxyethyl)-1H-pyrazol-4-yl]oxy}isophthalonitrile or pharmaceutically acceptable salts, solvates or derivatives thereof; GW-678248, GW-695634, MIV-150, calanolide, and tricyclic pyrimidinone derivatives as disclosed in WO 03/062238.

Examples of CCR5 antagonists include, but are not limited to, TAK-77; SC-351125; ancriviroc (formerly known as SCH-C; vicriviroc (formerly known as SCH-D); PRO-140; maraviroc; aplaviroc (formerly known as GW-873140, Ono-4128, AK-602); AMD-887; CMPD-167; methyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate or pharmaceutically acceptable salts, solvates or derivatives thereof; methyl 3-endo-{8-[(3S)-3-(acetamido)-3-(3-fluorophenyl)propyl]-8-azabicyclo[3.2.1]oct-3-yl}-2-methyl-4,5,6,7-tetrahydro-3H-imidazo[4,5-c]pyridine-5-carboxylate or pharmaceutically acceptable salts, solvates or derivatives thereof; ethyl 1-endo-{8-[(3S)-3-(acetylamino)-3-(3-fluorophenyl)propyl]-8-azabicyclo[3.2.1]oct-3-yl}-2-methyl-4,5,6,7-tetrahydro-1H-imidazo[4,5-c]pyridine-5-carboxylate or pharmaceutically acceptable salts, solvates or derivatives thereof; and N-{(1S)-3-[3-endo-(5-Isobutyryl-2-methyl-4,5,6,7-tetrahydro-1H-imidazo[4,5-c]pyridin-1-yl)-8-azabicyclo[3.2.1]oct-8-yl]-1-(3-fluorophenyl)propyl}acetamide) or pharmaceutically acceptable salts, solvates or derivatives thereof.

Examples of entry and fusion inhibitors include, but are not limited to, BMS-806, BMS-488043, 5-{(1S)-2-[(2R)-4-Benzoyl-2-methyl-piperazin-1-yl]-1-methyl-2-oxo-ethoxy}-4-methoxy-pyridine-2-carboxylic acid methylamide and 4-{(1S)-2-[(2R)-4-Benzoyl-2-methyl-piperazin-1-yl]-1-methyl-2-oxo-ethoxy}-3-methoxy-N-methyl-benzamide, enfuvirtide (T-20), sifuvirtide, P-01A, T1249, PRO 542, AMD-3100, soluble CD4, compounds disclosed in JP 2003171381, and compounds disclosed in JP 2003119137.

Examples of inhibitors of HIV integrase include, but are not limited to, L-000870810 GW-810781, 1,5-naphthyridine-3-carboxamide derivatives disclosed in WO 03/062204, compounds disclosed in WO 03/047564, compounds disclosed in WO 03/049690, and 5-hydroxypyrimidine-4-carboxamide derivatives disclosed in WO 03/035076, GS-9137 (JTK-303).

Examples of prenylation inhibitors include, but are not limited to, HMG CoA reductase inhibitors, such as statins (e.g. atorvastatin).

Examples of maturation inhibitors include 3-O-(3′,3′-dimethylsuccinyl) betulic acid (otherwise known as PA-457) and alpa-HGA.

Anti-infectives that may be used in combination with the compounds of the present invention include antibacterials and antifungals. Examples of antibacterials include, but are not limited to, atovaquone, azithromycin, clarithromycin, trimethoprim, trovafloxacin, pyrimethamine, daunorubicin, clindamycin with primaquine, fluconazole, pastill, ornidyl, eflornithine pentamidine, rifabutin, spiramycin, intraconazole-R51211, trimetrexate, daunorubicin, recombinant human erythropoietin, recombinant human growth hormone, megestrol acetate, testerone, and total enteral nutrition. Examples of antifungals include, but are not limited to, anidulafungin, C31G, caspofungin, DB-289, fluconazole, itraconazole, ketoconazole, micafungin, posaconazole, and voriconazole.

There is also included within the scope the present invention, combinations of a compound of formula (I), or a pharmaceutically acceptable salt, solvate or derivative thereof, together with one or more additional therapeutic agents independently selected from the group consisting of:

Proliferation inhibitors, e.g. hydroxyurea.

Immunomodulators, such as AD-439, AD-519, alpha interferon, AS-101, bropirimine, acemannan, CL246,738, EL10, FP-21399, gamma interferon, granulocyte macrophage colony stimulating factor (e.g. sargramostim), IL-2, immune globulin intravenous, IMREG-1, IMREG-2, imuthiol diethyl dithio carbamate, alpha-2 interferon, methionine-enkephalin, MTP-PE, remune, rCD4, recombinant soluble human CD4, interferon alfa-2, SK&F106528, soluble T4 thymopentin, tumor necrosis factor (TNF), tucaresol, recombinant human interferon beta, interferon alfa n-3.

Tachykinin receptor modulators (e.g. NK1 antagonists) and various forms of interferon or interferon derivatives.

Other chemokine receptor agonists/antagonists such as CXCR4 antagonists (e.g AMD070 and AMD3100) or CD4 antagonists (e.g. TNX-355).

Agents which substantially inhibit, disrupt or decrease viral transcription or RNA replication such as inhibitors of tat (transcriptional trans activator) or nef (negative regulatory factor).

Agents which substantially inhibit, disrupt or decrease translation of one or more proteins expressed by the virus (including, but not limited to, down regulation of protein expression or antagonism of one or more proteins) other than reverse transcriptase, such as Tat or Nef.

Agents which influence, in particular down regulate, CCR5 receptor expression; chemokines that induce CCR5 receptor internalisation such MIP-1α, MIP-1β, RANTES and derivatives thereof; examples of such agents include, but are not limited to, immunosupressants, such as calcineurin inhibitors (e.g. tacrolimus and cyclosporin A); steroids; agents which interfere with cytokine production or signalling, such as Janus Kinase (JAK) inhibitors (e.g. JAK-3 inhibitors, including 3-{(3R,4R)-4-methyl-3-[methyl-(7H-pyrrolo[2,3-d]pyrimidin-4-yl)-amino]-piperidin-1-yl}-3-oxo-propionitrile) and pharmaceutically acceptable salts, solvates or derivatives thereof; cytokine antibodies (e.g. antibodies that inhibit the interleukin-2 (IL-2) receptor, including basiliximab and daclizumab);

Agents which interfere with cell activation or cell cycling, such as rapamycin.

In addition to the requirement of therapeutic efficacy, which may necessitate the use of therapeutic agents in addition to the compounds of formula (I), there may be additional rationales which compel or highly recommend the use of a combination of a compound of the invention and another therapeutic agent, such as in the treatment of diseases or conditions which directly result from or indirectly accompany the basic or underlying CCR5 chemokine receptor modulated disease or condition. For example, where the basic CCR5 chemokine receptor modulated disease or condition is HIV infection and multiplication it may be necessary or at least desirable to treat Hepatitis C Virus (HCV), Hepatitis B Virus (HBV), Human Papillomavirus (HPV), neoplasms, and other conditions which occur as the result of the immune-compromised state of the patient being treated. Other therapeutic agents may be used with the compounds of formula (I), e.g., in order to provide immune stimulation or to treat pain and inflammation which accompany the initial and fundamental HIV infection.

Accordingly, therapeutic agents for use in combination with the compounds of formula (I) and their pharmaceutically acceptable salts, solvates and derivatives also include:

Agents useful in the treatment of hepatitis, such as interferons, pegylated interferons (e.g. peginterferon alfa-2a and peginterferon alfa-2b), long-acting interferons (e.g. albumin-interferon alfa); TLR7 inhibitors; reverse transcriptase inhibitors, such as lamivudine and emtricitabine; IMP dehydrogenase inhibitors such as ribavirin and viramidine; polymerase inhibitors (including NS5B polymerase inhibitors) such as valopicitabine, HCV-086, HCV-796 purine nucleoside analogues as disclosed in WO 05/009418, and imidazole derivatives as disclosed in WO 05/012288; alpha glucosidase inhibitors such as celgosivir; interferon enhancers such as EMZ-702; serine protease inhibitors such as BILN-2061, SCH-6, VX-950, aza-peptide-based macrocyclic derivatives as disclosed in WO 05/010029 and those disclosed in WO 05/007681; caspase inhibitors such as IDN-6566; HCV replicon inhibitors such as arylthiourea derivatives as disclosed in WO 05/007601.

Agents useful in the treatment of AIDS related Kaposi's sarcoma, such as interferons, daunorubicin, doxorubicin, paclitaxel, metallo-matrix proteases, A-007, bevacizumab, BMS-275291, halofuginone, interleukin-12, rituximab, porfimer sodium, rebimastat, COL-3.

Agents useful in the treatment of cytomegalovirus (CMV), such as fomivirsen, oxetanocin G, cidofovir, cytomegalovirus immune globin, foscarnet sodium, Isis 2922, valacyclovir, valganciclovir, ganciclovir.

Agents useful in the treatment of herpes simplex virus (HSV), such as acyclovir, penciclovir, famciclovir, ME-609.

Further combinations for use according to the invention include combination of a compound of formula (I), or a pharmaceutically acceptable salt, solvate or derivative thereof with a CCR1 antagonist, such as BX-471; a beta adrenoceptor agonist, such as salmeterol; a corticosteroid agonist, such fluticasone propionate; a LTD4 antagonist, such as montelukast; a muscarinic antagonist, such as tiotropium bromide; a PDE4 inhibitor, such as cilomilast or roflumilast; a COX-2 inhibitor, such as celecoxib, valdecoxib or rofecoxib; an alpha-2-delta ligand, such as gabapentin or pregabalin; a beta-interferon, such as REBIF; a TNF receptor modulator, such as a TNF-alpha inhibitor (e.g. adalimumab).

There is also included within the scope the present invention, combinations of a compound of formula (I), or a pharmaceutically acceptable salt, solvate or derivative thereof, together with one or more additional therapeutic agents which slow down the rate of metabolism of the compound of the invention, thereby leading to increased exposure in patients. Increasing the exposure in such a manner is known as boosting. This has the benefit of increasing the efficacy of the compound of the invention or reducing the dose required to achieve the same efficacy as an unboosted dose. The metabolism of the compounds of formula (I) includes oxidative processes carried out by P450 (CYP450) enzymes, particularly CYP 3A4 and conjugation by UDP glucuronosyl transferase and sulphating enzymes. Thus, among the agents that may be used to increase the exposure of a patient to a compound of the present invention are those that can act as inhibitors of at least one isoform of the cytochrome P450 (CYP450) enzymes. The isoforms of CYP450 that may be beneficially inhibited include, but are not limited to, CYP1A2, CYP2D6, CYP2C9, CYP2C19 and CYP3A4. Suitable agents that may be used to inhibit CYP 3A4 include, but are not limited to, ritonavir, saquinavir or ketoconazole.

In the above-described combinations, the compound of formula (I) or a pharmaceutically acceptable salt, solvate or derivative thereof and other therapeutic agent(s) may be administered, in terms of dosage forms, either separately or in conjunction with each other; and in terms of their time of administration, either simultaneously or sequentially. Thus, the administration of one component agent may be prior to, concurrent with, or subsequent to the administration of the other component agent(s).

Accordingly, in a further aspect the invention provides a pharmaceutical composition comprising a compound of formula (I) or a pharmaceutically acceptable salt, solvate or derivative thereof and one or more additional therapeutic agents.

The invention is illustrated by the following Preparations and Examples in which the following further abbreviations may be used:
0.88 ammonia=concentrated ammonium hydroxide solution
APCI=atmospheric pressure chemical ionisation
DMSO=dimethyl sulphoxide
ES=electrospray ionisation
HRMS=high resolution mass spectrum
LCMS=liquid chromatography-mass spectroscopy;
LRMS=low resolution mass spectrum
MS=mass spectrum
NMR=nuclear magnetic resonance
eq.=equivalent
RT=room temperature
h=hour
min=minute
m.p.=melting point

### EXAMPLE 1

### N-(3-chlorobenzyl)-N-{(3R)-1-[1-(2,6-dimethylbenzoyl)piperidin-4-yl]pyrrolidin-3-yl}cyclopropanecarboxamide

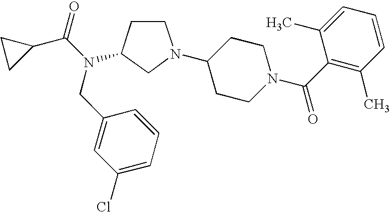

To a stirred solution of preparation 4 (200 mg, 0.66 mmol) in methylene chloride (3 ml) was added 3-chlorobenzaldehyde (83 ul, 0.7 mmol), sodium triacetoxyborohydride (211 mg) and acetic acid (38 μl). The reaction mixture was stirred at RT for 4 hours, treated with 1N NaOH (4 ml) and stirred for a further 10 minutes. The reaction mixture was filtered through a phase separation cartridge, washing with further dichloromethane.

The solvent was removed and the residue re-dissolved in DCM (3 ml) and treated with Et₃N (110 μl) and cyclopropane carbonyl chloride (64 μl). After stirring at RT overnight, the reaction mixture was quenched with 1N NaOH (3 ml) and after 10 minutes filtered through phase separation cartridges washing with further dichloromethane. The organic phase was concentrated and purified by Jones® parallel chromatography system eluting with a gradient 0-20% MeOH in DCM to afford the title compound as a white foam (137 mg).

¹H NMR (400 MHz, CD₃OD) δ 0.68-0.75 (1H, m), 0.86-0.95 (2H, m), 1.19-2.06 (6H, 8×m), 2.17 (3H, s), 2.27 (3H, s), 2.10-2.35 (1H, 2×m), 2.39-3.13 (7H, 5H×m), 4.42-5.11 (6H, 5×m), 7.05-7.09 (2H, m), 7.13-7.22 (3H, m), 7.24-7.40 (2H, 2×m).

Elemental analysis—observed C (69.86%), H (7.39%), N (8.43%); calculated for C₂₉H₃₆ClN₃O₂.0.04 CH₂Cl₂: C (70.11%), H (7.31%), N (8.45%).

LRMS: m/z APCI+494 [MH⁺].

Examples 2 to 57 may all be prepared according to the preparation for example 1 and scheme 1 where R⁵, X and R¹ are indicated in the following table. Thus the corresponding benzaldehyde optionally substituted by X would replace 3-benzaldehyde in example 1 (represented by R^{3A}CHO in scheme 1) and in example 16, 2-pyridylaldehyde would replace the 3-chlorobenzaldehyde. Similarly, the corresponding R⁵COCl compound would replace the cyclopropanecarbonyl chloride of example 1.

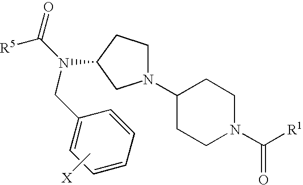

| | | | | LRMS |
|---|---|---|---|---|
| | | | | APCI |
| | | | | [MH+] |
| Example No. | R⁵ | X | R¹ | Data |
| 2 | 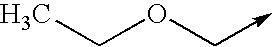 | H | 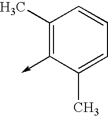 | 478 |
| 3 | 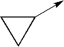 | 2-F | 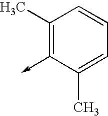 | 478 |
| 4 | 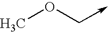 | H | 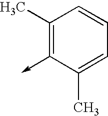 | 464 |
| 5 | 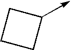 | H | 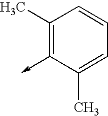 | 474 |
| 6 | 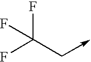 | H | 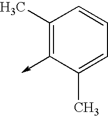 | 502 |
| 7 | 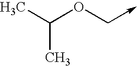 | H | 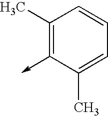 | 492 |
| 8 | 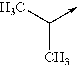 | H | 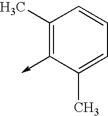 | 462 |
| 9 | 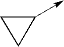 | 3-F | 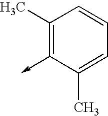 | 478 |
| 10 | 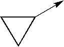 | H | 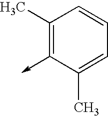 | 460 |
| 11 | 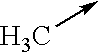 | H | 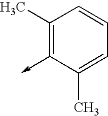 | 434 |
| 12 | 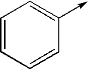 | H | 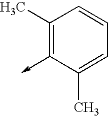 | 496 |
| 13 | 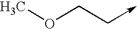 | H | 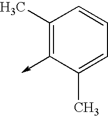 | 478 |
| 14 | 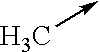 | 2-F | 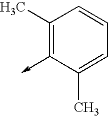 | 452 |
| 15 | 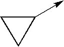 | 4-F | 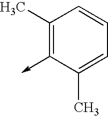 | 478 |
| 16 | 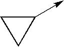 | 2- pyridyl | 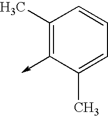 | 461 |
| 17 | 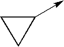 | H | 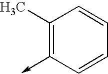 | 446 |
| 18 | 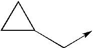 | H | 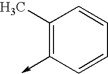 | 461 |
| 19 | 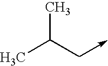 | H | 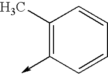 | 462 |
| 20 | 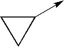 | H | 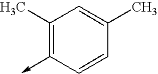 | 460 |
| 21 | 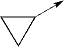 | H | 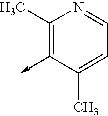 | 461 |
| 22 | 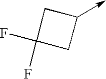 | H | 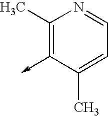 | 511 |
| 23 | 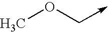 | 2,5- di F | 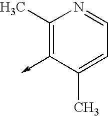 | 501 |
| 24 | 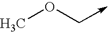 | 2-F | 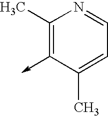 | 483 |
| 25 | 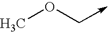 | 3-F | 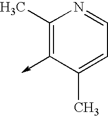 | 483 |
| 26 | 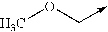 | 2,3- di F | 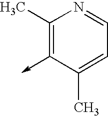 | 501 |
| 27 | 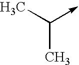 | 2-F | 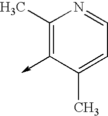 | 481 |
| 28 | 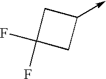 | 2-F | 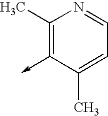 | 529 |
| 29 | 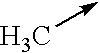 | H | 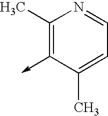 | 435 |
| 30 | 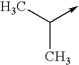 | H | 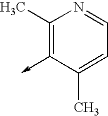 | 463 |
| 31 | 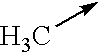 | H | 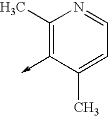 | 453 |
| 32 | 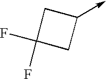 | 3-F | 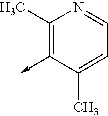 | 529 |
| 33 | 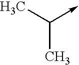 | 3-F | 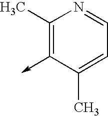 | 481 |
| 34 | 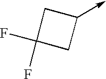 | H | 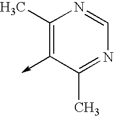 | 512 |
| 35 | 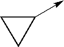 | H | 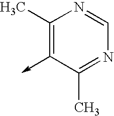 | 462 |
| 36 | 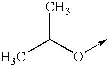 | H | 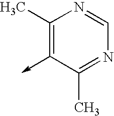 | 481 |
| 37 | 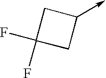 | 2-F | 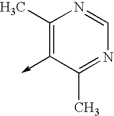 | 530 |
| 38 | 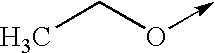 | H | 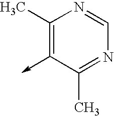 | 466 |
| 39 | 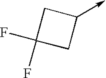 | 3-F | 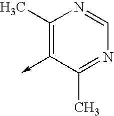 | 530 |
| 40 | 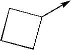 | H | 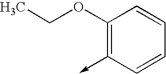 | 490 |
| 41 | 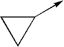 | H | 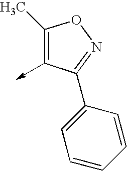 | 513 |
| 42 | 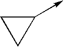 | H | 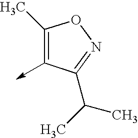 | 479 |
| 43 | 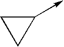 | H | 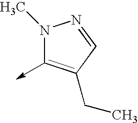 | 464 |
| 44 | 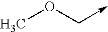 | 2-F | 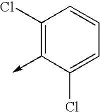 | 522 |
| 45 | 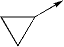 | H | 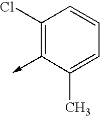 | 462 |
| 46 | 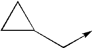 | H | 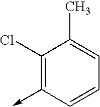 | 494 |
| 47 | 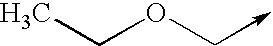 | H | 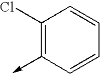 | 484 |
| 48 | 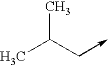 | H | 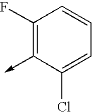 | 500 |
| 49 | 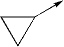 | H | 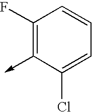 | 484 |
| 50 | 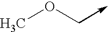 | 2-F | 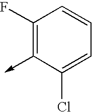 | 506 |
| 51 | 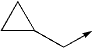 | H | 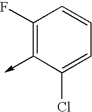 | 498 |
| 52 | 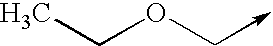 | H | 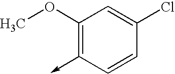 | 514 |
| 53 | 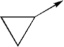 | H | 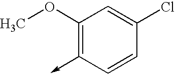 | 496 |
| 54 | 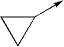 | H | 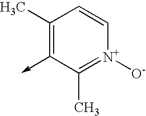 | 477 |
| 55 | 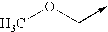 | H | 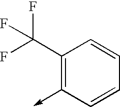 | 504 |
| 56 | 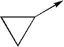 | H | 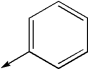 | 432 |
| 57 | 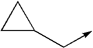 | H | 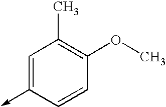 | 490 |

### EXAMPLE 58

### N-benzyl-N-{(3R)-1-[1-(2,6-dimethylbenzoyl)piperidin-4-yl]pyrrolidin-3-yl}methanesulfonamide

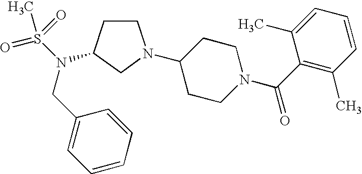

The title compound was prepared in accordance with example 1 except that CH₃SO₂Cl was used in place of cyclopropanecarbonyl chloride (step g in scheme 2). The title compound was obtained as a yellow foam (60 mg, 85%).

¹H NMR (400 MHz, CD₃OD) □ 1.2-1.5 (3H, m), 1.7-2.0 (3H, m), 2.18 (4H, m), 2.28 (4H, m), 2.4 (1H, q), 2.6-2.8 (4H, m), 2.9 (3H, s), 3.0 (2H, m), 4.4-4.6 (4H, m), 7.1 (2H, m), 7.2 (2H, m), 7.3 (2H, m), 7.4 (2H, m).

LRMS: m/z APCI+470 [MH⁺].

Elemental Analysis Observe 64.23 (C %), 7.41 (H %), 8.46 (N %); calc for 0.2DCM gives 64.58 (C %), 7.32 (H %), 8.62 (N %).

### EXAMPLE 59

### N-[(3R)-1-[1-(2,6-dimethylbenzoyl)piperidin-4-yl]pyrrolidin-3-yl]-N-(2-fluorobenzyl)-N′-propylurea

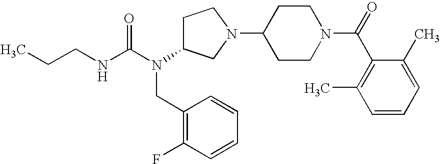

The title compound was prepared in accordance with example 1 except that the isocyanate CH₃(CH₂)NCO was used in place of cyclopropanecarbonyl chloride (step f in scheme 1). The title compound was obtained as a colourless oil (277 mg, 96%). The HCl salt was prepared by adding 10 ml of 1M HCl in Et₂O and scraping the oil to afford a white solid.

¹H NMR (400 MHz, CD₃OD) □ 0.86 (3H, t), 1.21-1.76 (6H, 3×m), 1.86-2.33 (6H, 3×m), 2.15 (3H, s), 2.24 (3H, s), 2.87-3.35 (6H, 4×m), 4.04 (1H, m), 4.52-4.66 (3H, m), 6.92-7.37 (8H, m).

LRMS: m/z APCI+495 [MH⁺].

Elemental Analysis Observe 64.86 (C %), 7.66 (H %), 10.28 (N %); calc for 1.0HCl gives 65.58 (C %), 7.59 (H %), 10.55 (N %).

### EXAMPLE 60

### N-benzyl-N-((3R)-1-{1-[(2,4-dimethylpyridin-3-yl)carbonyl]-3-methylpiperidin-4-yl}pyrrolidin-3-yl)cyclopropanecarboxamide

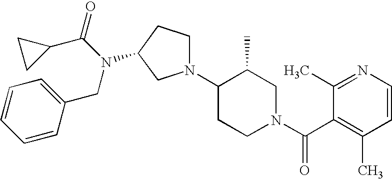

The title compound was prepared in accordance with example 1 except that the 1,4-dioxa-8-azaspiro(4.5)decane in preparation 1 was methyl substituted. The title compound was obtained as a white foam (143 mg, 64%).

¹H NMR (400 MHz, CDCl₃) □ 0.61-1.05 (8H, 5×m), 1.17-3.33 (9H, 7×m), 1.59 (3H, s), 2.17 & 2.39 (3H, 2×s), 2.33 & 2.55 (3H, 2×s), 4.62-4.90 (4H, m), 5.13-5.25 (1H, m), 6.91-7.36 (6H, 3×m), 8.32 (1H, m).

LRMS: m/z APCI+475 [MH⁺].

### EXAMPLE 61

### N-benzyl-N-{(3R)-1-[1-(2,6-dimethylbenzoyl)-4-methylpiperidin-4-yl]pyrrolidin-3-yl}cyclopropanecarboxamide

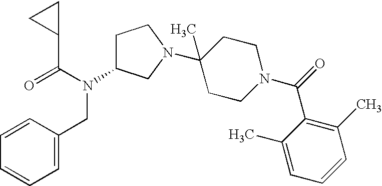

Titanium tetraisopropoxide (360 ul, 1.2 mmol) was added to a stirred solution of the title compounds of preparation 7 (200 mg, 0.8) and preparation 2 (200 mg, 0.9 mmol) at 0° C. and stirred overnight. The initially hazy solution became a clear orange solution which was concentrated in vacuo, taken up in toluene (7 ml), and treated with Et₂AlCN (1.1 ml, 1M in toluene, 1.1 mmol) before stirring at RT overnight. After diluting with EtOAc (10 ml) and water (0.4 ml) [caution], the mixture was stirred at RT for 1 h, filtered through Arbocel® and concentrated to an impure oil.

To the crude oil (360 mg, 0.7 mmol) in THF (6 ml) was slowly added MeMgBr (3M in Et₂O, 0.75 ml, 2.25 mmol). After 1 h at RT, the reaction mixture was carefully quenched with NH₄Cl (sat. aq.) and treated with EtOAc. The aq. phase was separated and extracted with EtOAc. Combined organics were washed with brine, dried (MgSO₄) and condensed to a residue which was purified by column chromatography (first silica, eluting DCM/MeOH 95/5, then on silica eluting DCM/MeOH/NH₄OH 96/4/0.4). The resultant product was portioned between EtOAc and aq. HCl, and the aq. Phase basified, and extracted with EtOAc. The extracts were combined, dried (MgSO₄) and condensed to give the title compound as a glass (150 mg, 45%).

¹H NMR (400 MHz, CDCl₃) □ 0.66 (2H, m), 0.92 (3H, s), 1.00 (2H, m), 1.20-1.87 (7H, 5×m), 2.09 (1H, m), 2.26 (6H, m), 2.44-2.81 (3H, 4×m), 2.96-3.53 (3H, m), 4.20 (1H, m), 4.86 (2H, m), 5.20 (1H, m), 7.02 (2H, m), 7.10-7.35 (6H, 3×m).

LRMS: m/z APCI+474 [MH⁺].

Examples 62 to 69 may all be prepared according to the preparation for example 62 where R⁵, X and R¹ are indicated in the following table.

Thus R⁵COCl would be used in place of cyclopropanecarbonyl chloride in preparation 6, R¹COOH would be used in place of 2,6 dimethylbenzoic acid in preparation 1. For examples 63, 65 and 68, 2F-substituted benzaldehyde would be used in place of benzaldehyde in preparation 5.

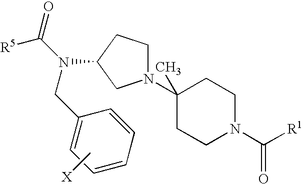

| | | | | LCMS |
|---|---|---|---|---|
| | | | | APCI |
| | | | | [MH+] |
| Example No. | R⁵ | X | R¹ | Data |
| 62 | 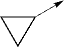 | H | 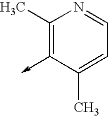 | 475 |
| 63 | 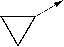 | H | 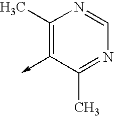 | 476 |
| 64 | 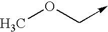 | 2-F | 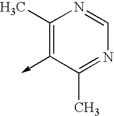 | 498 |
| 65 | 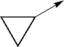 | H | 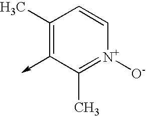 | 491 |
| 66 | 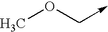 | 2-F | 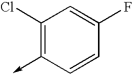 | 520 |
| 67 | 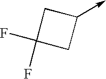 | H | 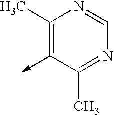 | 526 |
| 68 | 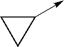 | H | 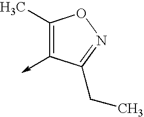 | 479 |
| 69 | 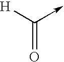 | 2-F | 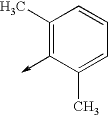 | 452 |

### EXAMPLE 70

### N-benzyl-N-((3R)-1-[(3-exo)-8-[(2,4-dimethylpyridin-3-yl)carbonyl]-8-azabicyclo[3.2.1]oct-3-yl]pyrrolidin-3-yl)cyclopropanecarboxamide

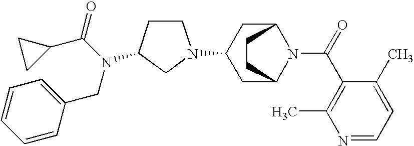

A solution of the product of preparation 9 (150 mg, 0.42 mmol), 2,4-dimethyl-3-pyridinecarboxylic acid (158 mg, 0.84 mmol), WSCDI (161 mg, 0.84 mmol), HOBT (113 mg, 0.84 mmol) and N-methylmorpholine (234□l, 1.68 mmol) in DCM (10 ml) was stirred for 16 h at RT, evaporated under reduced pressure to a gum, and partitoned between NaHCO₃ (3% aq., 3 ml) and EtOAc (5 ml). The aq. phase was separated, extracted with further EtOAc (5 ml), and combined organics were dried over Na₂SO₄ and evaporated to an oil. Purification by column chromatography (silica, eluting MeOH in DCM 0-8%) gave example 69 (exo isomer) as an oil (45 mg, 22%).

¹H NMR (400 MHz, CDCl₃) □ 0.56-0.90 (2H, m), 0.91-1.08 (2H, m), 1.12-3.01 (15H, 7×m), 2.20 & 2.42 (3H, 2×s), 2.36 & 2.59 (3H, 2×s), 3.55-3.64 (1H, m), 4.58-5.17 (4H, 4×m), 6.95-7.01 (1H, m), 7.13-7.37 (6H, 3×m), 8.35 (1H, m).

LRMS: m/z APCI+487 [MH⁺].

Examples 71 and 72 may all be prepared according to the preparation for example 70 where R⁵, X and R¹ are indicated in the following table.

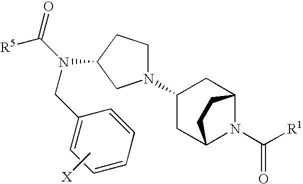

| | | | | LRMS |
|---|---|---|---|---|
| | | | | APCI |
| | | | | [MH+] |
| Example No. | R⁵ | C | R¹ | Data |
| 71 | 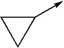 | H | 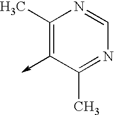 | 488 |
| 72 | 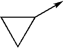 | H | 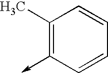 | 472 |

### Preparation 1

### 8-(2,6-dimethylbenzoyl)-1,4-dioxa-8-azaspiro[4.5]decane

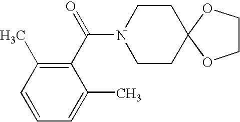

To a stirred solution of 2,6-dimethylbenzoic acid (2.50 g, 16.6 mmol) in DCM (90 ml) was added HOBT (2.29 g, 16.6 mmol), WSCDI (3.80 g, 19.9 mmol), N-methylmorpholine (3.66 ml, 33 mmol) and 1,4-dioxa-8-azaspiro(4.5)decane (2.38 g, 16.6 mmol). This was stirred for 16 h at RT and then the reaction was quenched by adding 1M aqueous sodium hydroxide solution (20 ml). The organic layer was separated, dried over magnesium sulfate and then evaporated to leave an orange oil. Purification by column chromatography (silica, eluting with MeOH in DCM 0-2%) afforded the title compound as a colourless oil (3.60 g, 79%).

LRMS: m/z APCI+276 [MH⁺].

### Preparation 2

### 1-(2,6-dimethylbenzoyl)piperidin-4-one

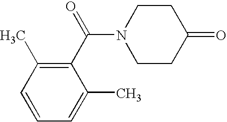

A solution of the product of preparation 1 (3.60 g, 13.1 mmol) in THF (35 ml) and 4N HCl (aq, 35 ml) was heated to 60° C. for 6 h, the THF removed in vacuo and the aq. residue extracted with EtOAc (3×30 ml). The organic phase was dried over magnesium sulfate and then evaporated to give an oil. Purification by column chromatography (silica, eluting with EtOAc in pentane 30-50%) afforded the title compound as a colourless oil (1.0 g, 33%).

LRMS: m/z APCI+232 [MH⁺].

### Preparation 3

### tert-butyl {(3R)-1-[1-(2,6-dimethylbenzoyl)piperidin-4-yl]pyrrolidin-3-yl}carbamate

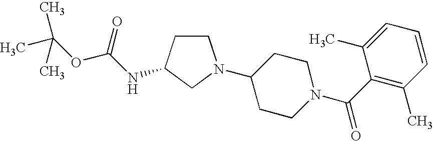

Commercially available (R)-pyrrolidin-3-yl-carbamic acid tert-butyl ester (3.0 g, 12 mmol), the title compound of preparation 2 (2.77 g, 12 mmol) and AcOH (0.69 ml, 12 mmol) in DCM (75 ml) was treated with sodium triacetoxyborohydride (3.82 g, 18 mmol). After 16 h at RT the reaction mixture was quenched by the addition of 1M aqueous NaOH solution. The separated organic phase was dried over MgSO₄ and then evaporated to give a white foam. Purification by column chromatography (silica, eluting with MeOH in DCM 5-10%) afforded the title compound as a white solid (5.0 g, 77%).

LRMS: m/z APCI+402 [MH⁺].

### Preparation 4

### (3R)-1-[1-(2,6-dimethylbenzoyl)piperidin-4-yl]pyrrolidin-3-amine

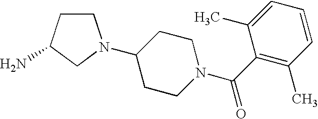

The title compound of preparation 3 (4.82 g, 12 mmol) was dissolved in 4M HCl in dioxan (24 ml) and stirred at RT for 3 h. The reaction mixture was concentrated under reduced pressure and then basified with 1M aqueous NaOH solution (20 ml) and then extracted with DCM (3×50 ml). The combined organics were dried over MgSO₄ and concentrated to give an orange oil. Purification by column chromatography (silica, eluting with MeOH in DCM 5-10%) afforded the title compound as a pale yellow oil (3.5 g, 96%).

LRMS: m/z APCI+302 [MH⁺].

### Preparation 5

### (3R)—N,1-dibenzylpyrrolidin-3-amine

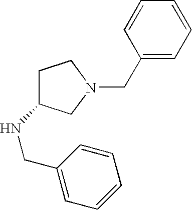

(R)-1-benzyl-pyrrolidin-3-ylamine (1.0 ml, 5.7 mmol), benzaldehyde (0.65 ml, 6.4 mmol) and sodium triacetoxyborohydride (1.84 g, 8.6 mmol) were taken up in acetic acid (0.33 ml, 5.7 mmol) and DCM (20 ml) and stirred at room temperature overnight under nitrogen. The mixture was washed with saturated aqueous NaHCO₃ solution and then dried (MgSO₄), evaporated and purified by flash column chromatography on silica gel using 5% methanol in DCM containing 0.5% ammonia solution as eluant to provide the title compound as a clear oil (1.20 g, 80%).

LRMS: m/z APCI+267 [MH⁺].

### Preparation 6

### N-benzyl-N-[(3R)-1-benzylpyrrolidin-3-yl]cyclopropanecarboxamide

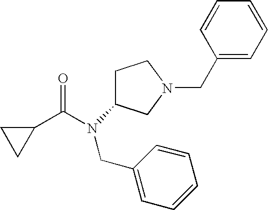

Cyclopropanecarbonyl chloride (0.4 ml, 4.4 mmol) was added dropwise to a solution of the title compound of preparation 5 (1.1 g, 4.1 mmol) and Et₃N (0.7 ml, 5 mmol) in DCM at RT. After 2 h, the reaction was quenched with NaHCO₃ (aq, sat.), dried (over MgSO₄) and concentrate to a residue which was purified by column chromatography (silica, eluting 99/1/0.1 to 981210.2 DCM/MeOH/NH₄OH) to afford the title compound as an oil (1.37 g, 99%).

LRMS: m/z APCI+335 [MH⁺].

### Preparation 7

### N-benzyl-N-[(3R)-pyrrolidin-3-yl]cyclopropanecarboxamide

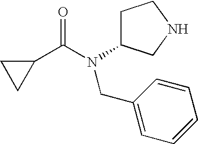

The product of preparation 6 (1.58 g, 4.73 mmol) was taken up in EtOH (50 ml) under nitrogen, ammonium formate (1.75 g, 27.8 mmol) and 20% Pd(OH)₂ on charcoal (0.15 g) were added and the whole was heated at 75° C. for 2 h. After cooling to RT, the mixture was filtered through a short plug of Arbocel and washed with EtOH (50 ml). The filtrate was evaporated to dryness and the residue purified by flash column chromatography (silica, eluting DCM/MeOH/NH₃ solution 90/10/1) to provide the title compound as a clear oil (986 mg, 85%).

LRMS: m/z APCI+245 [MH⁺].

### Preparation 8

### tert-butyl 3-[(3R)-3-[benzyl(cyclopropylcarbonyl)amino]pyrrolidin-1-yl]-8-azabicyclo[3.2.1]octane-8-carboxylate

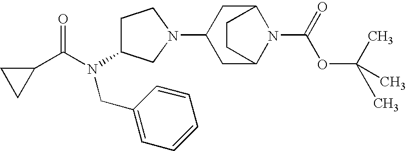

The title compound of preparation 7 (461 mg, 1.64 mmol), commercially available Boc-nortropinone (369 mg, 1.64 mmol) and AcOH (0.28 ml, 4.92 mmol) in DCM (20 ml) was treated with sodium triacetoxyborohydride (521 mg, 2.46 mmol). After 3 h at RT, the reaction mixture was quenched by the addition of saturated aqueous NaHCO₃ solution. The organic phase was separated, dried over MgSO₄ and evaporated to give an oil which was purified by column chromatography (silica, eluting 99/1/0.1 to 98/2/0.2 DCM/MeOH/NH₄OH) to afford the title compound as an oil (0.586 g, 79%).

LRMS: m/z APCI+455 [MH⁺].

### Preparation 9

### N-[(3R)-1-(8-azabicyclo[3.2.1]oct-3-yl)pyrrolidin-3-yl]-N-benzylcyclopropanecarboxamide

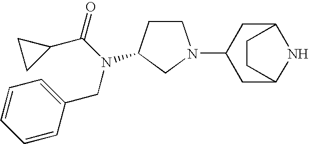

The product of preparation 8 (586 mg, 1.3 mmol) was dissolved in DCM (20 ml), and stirred with ethereal HCl (2M, 10 ml) overnight at RT. After evaporation of solvents, the residue was dissolved in DCM (10 ml), washed with saturated aqueous NaHCO₃ solution (10 ml) and the aqueous phase extracted with further DCM (5×10 ml). The combined organics were dried over MgSO₄, and purified by column chromatography (reverse-phase silica, eluting with a gradient from 100% water to 100% MeCN) to afford the title compound as a clear oil (301 mg, 66%).

LRMS: m/z APCI+355 [MH⁺].

### Biological Data

The ability of the compounds of formula (I) and their pharmaceutically acceptable salts, solvates and derivatives to modulate chemokine receptor activity is demonstrated by methodology known in the art, such as by using the assay for CCR5 binding following procedures disclosed in Combadiere et al., J. Leukoc. Biol., 60, 147-52 (1996); and/or by using the intracellular calcium mobilisation assays as described by the same authors, and/or inhibiting cell fusion following procedures disclosed in Bradley et al., J Biomol Screen 9, 516-24 (2004).

Cell lines expressing the receptor of interest include those naturally expressing the receptor, such as PM-1, or IL-2 stimulated peripheral blood lymphocytes (PBL), or a cell engineered to express a recombinant receptor, such as CHO, 300.19, L1.2 or HEK-293.

Of the examples tested, the compounds when tested using the assay for intracellular mobilisation according to Combadiere et al (ibid) were found to be potent CCR5 antagonists with IC50 values of less than 10 μM.

The pharmacological activity of the compounds of formula (I) and their pharmaceutically acceptable salts, solvates and derivatives is further demonstrated using a gp160 induced cell-cell fusion assay to determine the IC₅₀ values of compounds against HIV-1 fusion. The gp160 induced cell-cell fusion assay uses a HeLa P4 cell line and a CHO-Tat10 cell line.

The HeLa P4 cell line expresses CCR5 and CD4 and has been transfected with HIV-1 LTR-β-Galactosidase. The media for this cell line is Dulbecco modified eagle's medium (D-MEM) (without L-glutamine) containing 10% foetal calf serum (FCS), 2 mM L-glutamine, penicillin/streptomycin (Pen/Strep; 100 U/mL penicillin+10 mg/mL streptomycin), and 1 μg/ml puromycin.

The CHO cell line is a Tat (transcriptional trans activator)-expressing clone from a CHO JRR17.1 cell line that has been transfected with pTat puro plasmid. The media for this cell line is rich medium for mammalian cell culture originally developed at Roswell Park Memorial Institute RPMI1640 (without L-glutamine) containing 10% FCS, 2 mM L-glutamine, 0.5 mg/ml Hygromycin B and 12 μg/ml puromycin. The CHO JRR17.1 line expresses gp160 (JRFL) and is a clone that has been selected for its ability to fuse with a CCR5/CD4 expressing cell line.

Upon cell fusion, Tat present in the CHO cell is able to transactivate the HIV-1 long terminal repeat (LTR) present in the HeLa cell leading to the expression of the β-Galactosidase enzyme. This expression is then measured using a Fluor Ace™ β-Galactosidase reporter assay kit (Bio-Rad cat no. 170-3150). This kit is a quantitative fluorescent assay that determines the level of expression of β-galactosidase using 4-methylumbelliferyl-galactopyranoside (MUG) as substrate. β-Galactosidase hydrolyses the fluorogenic substrate resulting in release of the fluorescent molecule 4-methylumbelliferone (4 MU). Fluorescence of 4-methylumbelliferone is then measured on a fluorometer using an excitation wavelength of 360 nm and emission wavelength of 460 nm.

Compounds that inhibit fusion will give rise to a reduced signal and, following solubilisation in an appropriate solvent and dilution in culture medium, a dose-response curve for each compound can be used to calculate IC₅₀ values.

The compounds were found to be active in the HIV cell fusion assay:

| | Example No | | | | | | | | |
|---|---|---|---|---|---|---|---|---|---|
| | 1 | 2 | 3 | 4 | 5 | 6 | 7 | 8 | 9 |
| IC₅₀ | 0.14 | 0.11 | 0.20 | 0.23 | 0.27 | 0.34 | 0.42 | 0.63 | 0.80 |
| (Nm) | | | | | | | | | |
| | Example No | | | | | | | | |
| | 10 | 11 | 12 | 13 | 14 | 15 | 16 | 17 | 18 |
| IC₅₀ | 1.99 | 3.05 | 4.06 | 4.83 | 13.0 | 34.1 | 237 | 40.8 | 187 |
| (Nm) | | | | | | | | | |

| | Example No | | | | | | | | |
|---|---|---|---|---|---|---|---|---|---|
| | 19 | 20 | 21 | 22 | 23 | 24 | 25 | 26 | 27 |
| IC₅₀ | 285 | 221 | 0.82 | 1.21 | 1.42 | 119 | 5.04 | 6.51 | 8.8 |
| (Nm) | | | | | | | | | |

| | Example No | | | | | | | | |
|---|---|---|---|---|---|---|---|---|---|
| | 28 | 29 | 30 | 31 | 32 | 33 | 34 | 35 | 36 |
| IC₅₀ | 12.4 | 14.0 | 18.9 | 26.4 | 34.7 | 42.2 | 8.51 | 9.9 | 52.0 |
| (Nm) | | | | | | | | | |

| | Example No | | | | | | | | |
|---|---|---|---|---|---|---|---|---|---|
| | 37 | 38 | 39 | 40 | 41 | 42 | 43 | 44 | 45 |
| IC₅₀ | 62.6 | 135 | 227 | 120 | 0.41 | 66.3 | 59.8 | 1.84 | 85.7 |
| (Nm) | | | | | | | | | |
| | Example No | | | | | | | | |
| | 46 | 47 | 48 | 49 | 50 | 51 | 52 | 53 | 54 |
| IC₅₀ | 107.0 | 19.3 | 14.1 | 28.5 | 77.7 | 100.0 | 15.6 | 104.0 | 34.3 |
| (Nm) | | | | | | | | | |

| | Example No | | | | | | | | |
|---|---|---|---|---|---|---|---|---|---|
| | 55 | 56 | 57 | 58 | 59 | 60 | 61 | 62 | 63 |
| IC₅₀ | 48.1 | 1.9 | 6.2 | 92.5 | 0.02 | 4.46 | 0.23 | 0.34 | 8.18 |
| (Nm) | | μm | μm | | | | | | |

| | Example No | | | | | | | | |
|---|---|---|---|---|---|---|---|---|---|
| | 64 | 65 | 66 | 67 | 68 | 69 | 70 | 71 | 72 |
| IC₅₀ | 16.6 | 26.5 | 28.5 | 44.3 | 61.6 | 123.0 | 2.65 | 4.27 | 97.3 |
| (Nm) | | | | | | | | | |

1. A compound of formula (I) 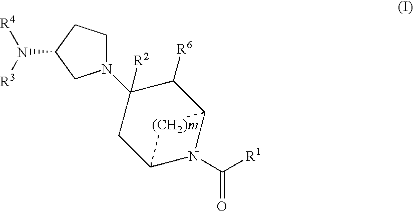
or a pharmaceutically acceptable salt, solvate of derivative thereof, wherein:
R¹ is aryl; or Het¹; and wherein the said aryl and Het₁ are substituted by 0 to 3 atoms or groups selected from C₁₋₆alkyl, C₃₋₇ cycloalkyl, C₁₋₆ alkoxy, C₁₋₆ alkoxyC₁₋₆ alkyl, halogen, C₁₋₆ haloalkyl, OH, CN, phenyl or imidazolyl;
R² is H or C₁₋₃ alkyl
R³ is C₁₋₆alkyl, C₃₋₇cycloalkyl, aryl, arylC₁₋₃alkyl, Het²C₁₋₃alkyl wherein the said aryl and Het² are substituted by 0 to 3 atoms or groups selected from C₁₋₆ alkyl, C₃₋₇ cycloalkyl, C₁₋₆ alkoxy, C₁₋₆ alkoxyC₁₋₆ alkyl, halogen, C₁₋₆ haloalkyl, OH or CN
R⁴ is COR⁵ or SO₂R⁵;
R⁵ is H, aryl, arylC₁₋₃alkyl, C₁₋₆alkyl, C₃₋₇cycloalkyl, C₃₋₇cycloalkylC₁₋₃alkyl, C₁₋₆alkoxy, C₁₋₆alkoxyC₁₋₆alkyl, C₀₋₆alkylaminoC₀₋₆alkyl or a 5 to 6 membered saturated heterocycle containing one to three heteroatoms selected from N, O and S; wherein the said C₁₋₆ alkyl, C₃₋₇ cycloalkyl, C₃₋₇cycloalkylC₁₋₃alkyl, C₁₋₆ alkoxy, C₁₋₆ alkoxyC₁₋₆ alkyl and C₀₋₆alkylaminoC₀₋₆alkyl are substituted by 0 to 3 atoms or groups selected from halogen, C₁₋₆ alkoxy or OH;
R⁶ is H or C₁₋₄alkyl;
m is 0, 1, 2 or 3;
with the proviso that when m is 1, 2 or 3 then R⁶ is H
“-----” represents an optionally present C—C bond such that, when m=1, 2 or 3, any two of the bonds are present in the piperidine ring to form an alkylene bridge.
Het¹ is a 5 to 10-membered aromatic heterocycle containing one to three heteroatoms selected from N, O and S, and wherein when Het¹ is a N-containing heterocycle, N-oxides thereof;
Het² is a 5 or 6 membered aromatic heterocycle containing one to three heteroatoms selected from N, O and S, and wherein when Het² is a N-containing heterocycle, N-oxides thereof.

2. The compound as claimed in claim 1 wherein Het¹ is a 5 to 6 membered heterocycle containing 1 to 3 heteroatoms selected from N, O and S and wherein when Het¹ is a N-containing heterocycle, N-oxides thereof, substituted as in claim 1.

3. (canceled)

4. (canceled)

5. The compound as claimed in claim 1 wherein R¹ is phenyl, pyridyl, pyrimidyl, pyridyl N-oxide or pyrimidyl N-oxide substituted with 1 or 2 atoms or groups selected from C₁₋₃alkyl, C₁₋₆ alkoxy or halogen.

6. The compound as claimed in claim 1 wherein R¹ is 2,6-dimethylphenyl, 2,4-dimethylpyridin-3-yl or 4,6-dimethylpyrimidin-5-yl.

7. The compound as claimed in claim 1 wherein R² is H.

8. The compound as claimed in claim 1 wherein R³ is benzyl, pyridylmethyl or pyrimidylmethyl substituted as defined in claim 1.

9. The compound as claimed in claim 6 wherein R³ is benzyl substituted by 0 to 3 atoms or groups selected from C₁₋₃alkyl, halogen, C₁₋₃alkoxy, or C₁₋₃haloalkyl.

10. The compound as claimed in claim 9 wherein the benzyl is substituted by 0 to 2 fluorine or chlorine atoms.

11. The compound as claimed in claim 1 wherein R⁴ is COR⁵ or SO₂R⁵ and R⁵ is H, phenyl, C₁₋₆alkyl, C₃₋₇cycloalkyl, C₃₋₇cycloalkylmethyl, C₁₋₃alkoxy, C₁₋₃ alkoxyC₁₋₃ alkyl or C₁₋₆ alkylamino, wherein the C₁₋₆alkyl, C₃₋₇cycloalkyl, C₃₋₇cycloalkylmethyl, C₁₋₃alkoxy, C₁₋₃ alkoxyC₁₋₃ alkyl or C₁₋₆ alkylamino are substituted by 0 to 3 atoms or groups selected from halogen, C₁₋₆alkoxy or OH.

12. The compound as claimed in claim 11 wherein said substitution is by 0 to 3 halogen.

13. The compound as claimed in claim 12 wherein R⁵ is C₃₋₇ cycloalkyl, C₁₋₃ alkoxyC₁₋₃ alkyl or C₁₋₄ alkylamino wherein the cycloalkyl is substituted with 0 to 2 fluoro atoms.

14. The compound as claimed in claim 1 wherein R⁴ is COR⁵.

15. The compound as claimed in claim 1 wherein R⁶ is H.

16. (canceled)

17. The A compound as claimed in claim 1 wherein m is 0.

18. A pharmaceutical composition including a compound of formula (I) or a pharmaceutically acceptable salt or solvate thereof, according to claim 1, together with one or more pharmaceutically acceptable excipients, diluents or carriers.

19. A pharmaceutical composition according to claim 18 including one or more additional therapeutic agents.

20. (canceled)

21. (canceled)

22. (canceled)

23. (canceled)

24. (canceled)

25. (canceled)

26. A method of treatment of a mammal suffering from a disorder in which the modulation of CCR5 receptors is implicated which comprises treating said mammal with an effective amount of a compound of formula (I) or a pharmaceutically acceptable salt, solvate or derivative thereof according to any of claim 1.

27. The method according to claim 26, wherein the disorder is HIV, a retroviral infection genetically related to HIV, or AIDS.

28. The method according to claim 26, wherein the disorder is an inflammatory disease, an autoimmune disease or pain.

29. The method according to claim 26, wherein the disorder is rheumatoid arthritis, graft rejection, fibrosis or pain.
